(19) United States
(12) Patent Application Publication
(10) Pub. No.: US 2020/0060268 A1
(43) Pub. Date: Feb. 27, 2020

(54) BACTERICIDE COMPOSITION

(71) Applicant: KAO CORPORATION, Tokyo (JP)

(72) Inventors: Takuji KAWAMURA, Wakayama-shi (JP); Keigo SUZUKI, Wakayama-shi (JP); Shinya SAITO, Wakayama-shi (JP)

(73) Assignee: KAO CORPORATION, Tokyo (JP)

(21) Appl. No.: 16/609,839

(22) PCT Filed: Dec. 14, 2018
(86) PCT No.: PCT/JP2018/046014

§ 371 Date: Oct. 31, 2019

(30) Foreign Application Priority Data
Dec. 25, 2017 (JP) 2017-248120

(51) Int. Cl. A01N 33/16; A01N 33/04; A61L 2/18; A01N 25/30

(52) U.S. Cl. CPC A01N 33/16; A01N 25/30; A61L 2/18; A01N 33/04

(57) ABSTRACT

The present invention is a bactericide composition containing: (a) an alkyl or an alkenyl amine oxide having an alkyl group or an alkenyl group with 7 or more and 18 or less carbons; and (b) a mono- or polyoxyalkylene alkyl or alkenyl amine having an alkyl group or an alkenyl group with 8 or more and 24 or less carbons and having an average number of added moles of the oxyethylene group of one or more and 8 or less.

### FIELD OF THE INVENTION

The present invention relates to a bactericide composition, a sterilization method, and a method for enhancing a bactericidal effect of a bactericide composition.

### BACKGROUND OF THE INVENTION

In recent years, application of bactericide compositions to household compositions and personal care compositions has been actively performed due to an increase in hygiene awareness.

As such bactericide compositions, JP-A 11-246310 discloses a bactericide effective for sterilizing Legionella bacteria including a specific amine oxide as an active ingredient. In addition, JP-A 2017-88903 discloses a liquid disinfecting composition including a specific amine oxide-type surfactant having a linear alkyl group and a specific metal salt at a predetermined ratio.

On the other hand, as a detergent having sterilization performance, JP-A 2016-84422 discloses a liquid detergent composition containing a specific amine oxide-type surfactant and a specific nonionic surfactant.

### SUMMARY OF THE INVENTION

The amine oxide-type surfactant is not only effective as a bactericide, but also has high emulsifying capability and is expected to have a cleaning function for developing detergency against light oil stains, thus it is considered to be an ideal component to be applied to a bactericide composition. However, it has a problem that the bactericidal effect is low compared to bactericides such as a normal cationic surfactant, and effective bactericidal effect cannot be obtained particularly at a low concentration. Thus, a technique for enhancing the bactericidal effect of the amine oxide-type surfactant is required.

The present invention provides a bactericide composition containing an amine oxide-type surfactant and having an excellent bactericidal effect.

The present invention relates to a bactericide composition containing: (a) an alkyl or an alkenyl amine oxide having an alkyl group or an alkenyl group with 7 or more and 18 or less carbons; and (b) a mono- or polyoxyalkylene alkyl or alkenyl amine having an alkyl group or an alkenyl group with 8 or more and 24 or less carbons and including oxyethylene group as an oxyalkylene group, wherein an average number of added moles of the oxyethylene group is one or more and 8 or less.

Also, the present invention relates to a method for enhancing a bactericidal effect of a bactericide composition, wherein the bactericidal effect of the bactericide composition is enhanced by adding, to (a) an alkyl or an alkenyl amine oxide having an alkyl group or an alkenyl group with 7 or more and 18 or less carbons; and (B) a mono- or polyoxyalkylene alkyl or alkenyl amine having an alkyl group or an alkenyl group with 8 or more and 24 or less carbons and including oxyethylene group as an oxyalkylene group, wherein an average number of added moles of the oxyethylene group is one or more and 8 or less.

Also, the present invention relates to a sterilization method, wherein the bactericide composition of the present invention is brought into contact with a hard surface.

Hereinbelow, explanation will be provided while (a) an alkyl or an alkenyl amine oxide having an alkyl group or an alkenyl group with 7 or more and 18 or less carbons is referred to as component (a); and (b) a mono- or polyoxyalkylene alkyl or alkenyl amine having an alkyl group or an alkenyl group with 8 or more and 24 or less carbons and including oxyethylene group as an oxyalkylene group, wherein an average number of added moles of the oxyethylene group is one or more and 8 or less is referred to as component (b).

According to the present invention, a bactericide composition containing an amine oxide-type surfactant and excellent in bactericidal effect is provided. The bactericide composition of the present invention can provide an excellent bactericidal effect even when treated at a low concentration.

### EMBODIMENTS OF THE INVENTION

### [Bactericide Composition]

Amine oxide-type surfactants such as component (a) are known to have a bactericidal action. However, the bactericidal effect is difficult to be developed at a low concentration and thus, a satisfactory bactericidal effect cannot be obtained. The present invention has discovered that the effect of the amine oxide of component (a) can be synergistically improved by use of a predetermined amine compound as component (b) in combination. While the reason therefor is not certain, it is considered that it is because the composite of component (a) and component (b) effectively acts on the surface of hydrophobic bacteria. It has conventionally not been known in the field that the bactericidal effect is synergistically improved when component (b) of the present invention is used with component (a).

In the present invention, component (a) may be a bactericide. In addition, component (b) may be a component to enhance the bactericidal effect of component (a), that is, a bactericidal efficacy enhancer. Here, the bactericidal efficacy enhancer itself may not develop a bactericidal effect, or the effect thereof may be extremely low even if developed. Therefore, the present invention may be a bactericide composition containing component (a) being a bactericide and component (b) being a bactericidal efficacy enhancer.

<Component (a)>

Component (a) of the present invention is an alkyl or an alkenyl amine oxide having an alkyl group or an alkenyl group with 7 or more and 18 or less carbons.

The number of carbons of the alkyl group or the alkenyl group of component (a) is, from the viewpoint of the bactericidal effect, preferably 9 or more and more preferably 11 or more, and preferably 16 or less and more preferably 14 or less. Component (a) preferably has an alkyl group with 7 or more and 18 or less carbons.

Component (a) is, from the viewpoint of the bactericidal effect, suitably a compound represented by the following general formula (a1):

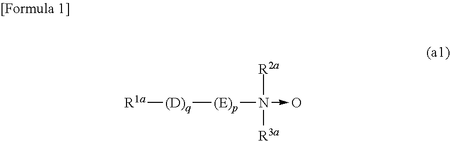

wherein R^{1a} represents an alkyl group or an alkenyl group, preferably an alkyl group, with 7 or more and 18 or less carbons, and R^{2a} and R^{1a} each independently represent an alkyl group with 1 or more and 3 or less carbons. D represents an —NHC(═O)— group or a —C(═O) NH— group, and E represents an alkylene group with one or more and 5 or less carbons. q and p represent q=0 and p=0, or q=1 and p=1.

In the above general formula (a1), R^{1a} is, from the viewpoint of the bactericidal effect, an alkyl group with preferably 9 or more and more preferably 11 or more carbons, and preferably 16 or less and more preferably 14 or less carbons. R^{2a} and R^{3a} are, from the viewpoint of the bactericidal effect, preferably a methyl group with one carbon. q and p are, from the viewpoint of the bactericidal effect, preferably q=0 and p=0. In addition, when q=0 and p=0, R^{1a} is an alkyl group with preferably 10 or more and more preferably 12 or more carbons, and preferably 16 or less and more preferably 14 or less carbons.

Preferable specific examples of component (a) include:

(1) a long-chain alkyl (having 8 or more and 18 or less carbons) di-short-chain alkyl (having one or more and 3 or less carbons) amine oxide such as lauryldimethylamine oxide; and
(2) a fatty acid (having 8 or more to 18 or less carbons) amidopropyl di-short-chain alkyl (having one or more and 3 or less carbons) amine oxide such as lauric acid amidopropyl dimethylamine oxide,
and from the viewpoint of the bactericidal effect, (1) a long-chain alkyl (having 8 or more and 18 or less carbons) di-short-chain alkyl (having one or more and 3 or less carbons) amine oxide is more preferable.
<Component (b)>

Component (b) of the present invention is a mono- or polyoxyalkylene alkyl or alkenyl amine having an alkyl group or an alkenyl group with 8 or more and 24 or less carbons and including oxyethylene group as an oxyalkylene group, wherein an average number of added moles of the oxyethylene group is one or more and 8 or less.

In component (b), the number of carbons of the alkyl group or the alkenyl group is, from the viewpoint of enhancing the bactericidal effect of component (a), 8 or more, preferably 10 or more and more preferably 12 or more, and 24 or less, preferably 22 or less, more preferably 20 or less, further preferably 18 or less and furthermore preferably 16 or less, and among these, the alkyl group is preferable.

Examples of the alkyl group or the alkenyl group with 8 or more and 24 or less carbons include a dodecyl group, a hexadecyl group, a heptadecyl group, an octadecyl group, and the like. These may be normal or iso, and may be a secondary alkyl group or alkenyl group. Here, the secondary alkyl group or alkenyl group refers to an alkyl group or an alkenyl group in which a carbon atom bonded to a nitrogen atom of amine is a tertiary carbon atom.

Component (b) has an oxyalkylene group, and includes, as the oxyalkylene group, an oxyethylene group with an average number of added moles of one or more and 8 or less. The average number of added moles of the oxyethylene group of component (b) is, from the viewpoint of enhancing the bactericidal effect of component (a), one or more and preferably 2 or more, and 8 or less and preferably 6 or less.

Also, in component (b), an oxyalkylene group other than the oxyethylene group, for example, an oxypropylene group, may be added within the range that does not impair the effect of the present invention. In component (b), an average number of added moles of the oxypropylene group is preferably 0.5 or less, and preferably 0 or more, and preferably 0. In component (b), the proportion of the oxyethylene group in all oxyalkylene groups is preferably 50 mol % or more, more preferably 80 mol % or more, further preferably 90 mol % or more, and further preferably 94 mol % or more and 100 mol % or less, and furthermore preferably 100 mol %.

Component (b) is, from the viewpoint of enhancing the bactericidal effect of component (a), suitably a compound represented by the following general formula (b1):

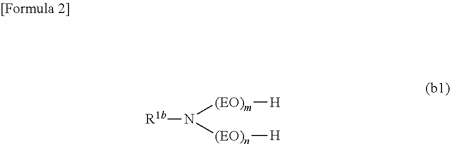

wherein R^{1b} is an alkyl group or an alkenyl group with 8 or more and 24 or less carbons, EO is an oxyethylene group, m and n are an average number of added moles and independently a number of 0 or more and 8 or less, and m+n is a number of one or more and 8 or less.

In the above general formula (b1), R^{1b} is, from the viewpoint of enhancing the bactericidal effect of component (a), an alkyl group or an alkenyl group with 8 or more, preferably 10 or more and more preferably 12 or more carbons, and 24 or less, preferably 22 or less, more preferably 20 or less, and further preferably 18 or less carbons. Also, R^{1b} is preferably an alkyl group. m and n are, from the viewpoint of enhancing the bactericidal effect of component (a), numbers in which m+n is one or more and preferably 2 or more, and 8 or less and preferably 6 or less.

### <Composition, Optional Component, Etc.>

The bactericide composition of the present invention contains component (a) in an amount of, from the viewpoint of the bactericidal effect, preferably 0.00005% by mass or more, more preferably 0.0001% by mass or more, further preferably 0.0005% by mass or more and furthermore preferably 0.001% by mass or more, and from the viewpoint of economic efficiency, preferably 5% by mass or less, more preferably 2% by mass or less, further preferably 1% by mass or less, furthermore preferably 0.5% by mass or less and furthermore preferably 0.1% by mass or less.

The bactericide composition of the present invention contains component (b) in an amount of, from the viewpoint of enhancing the bactericidal effect of component (a), preferably 0.00005% by mass or more, more preferably 0.0001% by mass or more, further preferably 0.0005% by mass or more and furthermore preferably 0.001% by mass or more, and from the viewpoint of economic efficiency, preferably 5% by mass or less, more preferably 2% by mass or less, further preferably 1% by mass or less, furthermore preferably 0.5% by mass or less and furthermore preferably 0.1% by mass or less.

In the bactericide composition of the present invention, a mass ratio (a)/(b) of the content of component (a) and the content of component (b) is, from the viewpoint of the bactericidal effect, preferably 0.001 or more, more preferably 0.01 or more and further preferably 0.1 or more, and from the same viewpoint, preferably 10 or less, more preferably 5 or less, more preferably 3 or less, further preferably 2 or less, furthermore preferably 1.5 or less and furthermore preferably 1 or less.

The bactericide composition of the present invention contains component (a) and component (b) in total in an amount of, from the viewpoint of the bactericidal effect, preferably 0.0001% by mass or more, more preferably 0.0005% by mass or more, further preferably 0.001% by mass or more and furthermore preferably 0.005% by mass or more, and from the viewpoint of economic efficiency, preferably 10% by mass or less, more preferably 5% by mass or less, further preferably 1% by mass or less and furthermore preferably 0.1% by mass or less.

In the bactericide composition of the present invention, from the viewpoint of the bactericidal effect, the ratio of the total of the content of component (a) and the content of component (b) relative to the total content of surfactants is preferably 70% by mass or more, more preferably 80% by mass or more, further preferably 85% by mass or more and furthermore preferably 90% by mass or more, and preferably 100% by mass or less; and may be 100% by mass.

The bactericide composition of the present invention preferably contains water. A liquid composition containing water is preferred. Water is contained in an amount that makes up the balance of the composition.

The bactericide composition of the present invention has, when in use, a pH at 20° C. (glass electrode method) of preferably 2 or more, more preferably 3 or more, further preferably 4 or more and furthermore preferably 5 or more, and preferably 12 or less, more preferably 11 or less, further preferably 10 or less and furthermore preferably 9 or less, from the viewpoint of stability, safety, usability and the like.

The present invention has found that component (b) enhances the bactericidal effect of component (a) which is difficult to be exhibited at a low concentration. The degree of enhancement of the bactericidal effect may vary depending on the type, concentration, ratio and the like of component (a) and component (b), but in any case, the present invention is the first to discover that the bactericidal effect of component (a) is enhanced by component (b). Also, the bactericide composition of the present invention may be a bactericide composition obtained by formulating component (a) and component (b).

The bactericide composition of the present invention is preferably for a hard surface of a hard article, and further for a hard surface of a food processing installation or a cooking installation. “Food processing installation and/or a cooking installation” means equipment and facility used in processing and/or cooking food in food processing factories. Examples of such equipment include food production or cooking equipment such as pipes, parts, net conveyors, freezers, slicers, and rice mills. Also, examples of such facilities include a floor, a wall, and a work bench.

Specifically, the bactericide composition of the present invention is preferably a bactericide for application to a hard surface of a hard article, more preferably a bactericide for application to a hard surface of a food processing installation or a cooking installation.

Further, specifically, the bactericide composition of the present invention is preferably a bactericide for spraying to a hard surface of a hard article, more preferably a bactericide for spraying to a hard surface of a food processing installation or a cooking installation.

As a hard article, food production equipment such as pipes, parts, and the like, hard articles around the kitchen, hard articles around the kitchen are articles used around the kitchen, and examples thereof specifically include:

(1) Storage locations of food, tableware, and cooking device such as refrigerator and cupboard,
(2) Cooking places of food such as drains, kitchen tables, range hoods, sinks, gas ranges, microwave ovens, and
(3) Floor and walls, etc. in the surrounding.
In the present invention, these are referred to as “hard articles around the kitchen” for convenience.

Also, examples of a hard surface, further a hard surface around the kitchen to be sterilized by the present invention include plastics (including a silicone resin and the like), metal, earthenware, wood, and combinations thereof.

### [A Method for Enhancing Bactericidal Effect of Bactericide Composition]

The present invention provides a method for enhancing a bactericidal effect of a bactericide composition, wherein the bactericidal effect of the bactericidal composition is enhanced by adding component (b) to a bactericide composition containing component (a). That is, component (b) functions as a bactericidal effect enhancer of component (a). The present invention provides a bactericidal effect enhancer of component (a) consisting of component (b). Also, the present invention provides a method for enhancing a bactericidal effect of component (a) by component (b). For these aspects of the present invention, matters described for the bactericide composition of the present invention can be appropriately applied.

### [Sterilization Method]

The present invention provides a sterilization method wherein the bactericide composition of the present invention is brought into contact with an object, preferably a hard surface, for sterilization thereof.

The sterilization method of the present invention can be suitably performed as a method for sterilizing a hard surface, wherein the bactericide composition of the present invention is brought into contact with the hard surface with oil spots including a degenerated oil, in particular a thermally degenerated oil attached thereto. Hereinbelow, a method for sterilizing a hard surface will be explained.

In the method for sterilizing a hard surface of the present invention, the bactericide composition is brought into contact with a hard surface. Specifically, preferably exemplified is a sterilization method in which the bactericidal composition as an undiluted solution is brought into contact with a hard surface, or the bactericidal composition as an undiluted solution is brought into contact with a hard surface without being diluted, that is, the bactericidal composition is brought into contact with a hard surface without being diluted. Further, exemplified is a sterilization method in which the bactericide composition is brought into contact with a hard surface with oil spots including a degenerated oil, in particular a thermally degenerated oil attached thereto, without being diluted. Since component (a) of the present invention has high emulsifying capability, it is possible to obtain an excellent bactericidal effect even on hard surfaces with oil spots attached thereto.

It should be noted that a concentrated composition containing components (a) and (b) of the present invention may be prepared, the concentrated composition may be diluted with water to prepare the bactericide composition of the present invention, and the bactericide composition may then be brought into contact with a hard surface. That is, it may be a method for sterilizing a hard surface wherein a concentrated composition containing component (a) and component (b) of the present invention is diluted with water to prepare the bactericide composition of the present invention and the bactericide composition is then brought into contact with a hard surface without being diluted.

In the present invention, the bactericide composition to be brought into contact with an object, for example a hard surface, contains component (a) in an amount of preferably 0.00005% by mass or more, more preferably 0.0001% by mass or more, further preferably 0.0005% by mass or more and furthermore preferably 0.001% by mass or more, and preferably 1% by mass or less, more preferably 0.5% by mass or less and further preferably 0.1% by mass or less.

In the present invention, the bactericide composition to be brought into contact with an object, for example a hard surface, contains component (b) in an amount of preferably 0.00005% by mass or more, more preferably 0.0001% by mass or more, further preferably 0.0005% by mass or more and furthermore preferably 0.001% by mass or more, and preferably 1% by mass or less, more preferably 0.5% by mass or less and further preferably 0.1% by mass or less.

In the present invention, the bactericide composition to be brought into contact with an object, for example, a hard surface, preferably has a mass ratio (a)/(b) in the above range.

Further, in the present invention, the bactericide composition to be brought into contact with an object, for example a hard surface, preferably also has a pH in the above range. In this case, the pH may be a pH at the temperature of the bactericide composition to be contacted with.

In addition, the method for sterilizing a hard surface of the present invention includes a sterilization method, which includes bringing the bactericide composition into contact with a hard surface with oil spots attached thereto and then, allowing to stand without applying (imparting) an external force such as a mechanical force. Since component (a) having a high emulsifying capability is contained, a high bactericidal effect can be obtained even when the composition is brought into contact without using a flexible material such as a sponge or fingers and allowed to stand as it is without applying an external force. This is preferable for sterilization of a site, to which a hand or a tool cannot reach, or a detail, which is difficult to reach.

Allowing to stand without applying an external force such as a mechanical force is, for example, to perform no intentional operation for sterilization other than the contact with the composition. For example, gravity flow of the contacted composition on a hard surface or propagation of vibrations not intended for sterilization on a hard surface can be understood as standing without applying an external force.

After allowing to stand, water is usually used for rinsing. During rinsing, an external force (physical force) by hand may be applied, or rinsing is carried out simply by a stream of water.

In the method for sterilizing a hard surface of the present invention, the bactericide composition is brought into contact with the hard surface as an object in a ratio of preferably 0.1 g or more, more preferably 0.3 g or more, and further preferably 0.4 g or more; and preferably 5 g or less, more preferably 3 g or less, and further preferably 2 g or less relative to 100 cm² of the hard surface, and further application or spraying is preferred.

In the method for sterilizing a hard surface of the present invention, from the viewpoint of increasing the bactericidal effect, after the bactericide composition is brought into contact with the hard surface, the hard surface is allowed to stand for preferably 10 seconds or more, more preferably 20 seconds or more, further preferably 30 seconds or more, and furthermore preferably 40 seconds or more; and from the same viewpoint, preferably 60 minutes or less, more preferably 30 minutes or less, further preferably 20 minutes or less, furthermore preferably 10 minutes or less, and furthermore preferably 5 or less. In this case, the time when the composition is first brought into contact with the hard surface may be regarded as a start of standing.

The temperature during standing may be room temperature, and for example, it is 10° C. or higher and 30° C. or lower.

In addition, in the method for sterilizing a hard surface of the present invention, from the viewpoint of increasing the bactericidal effect, the bactericide composition and the hard surface come into contact with each other for preferably 10 seconds or more, more preferably 20 seconds or more, further preferably 30 seconds or more, and furthermore preferably 40 seconds or more; and from the same viewpoint, preferably 60 minutes or less, more preferably 30 minutes or less, further preferably 20 minutes or less, furthermore preferably 10 minutes or less, and furthermore preferably 5 minutes or less.

In the method for sterilizing a hard surface of the present invention, the bactericide composition may be brought into contact with a hard surface with oil spots containing a degenerated oil, in particular a thermally degenerated oil attached thereto, by immersing the hard surface in the composition. However, from the viewpoint of efficiently increasing the bactericidal effect, preferred is a method of bringing the bactericide composition into contact with the hard surface with oil spots containing a degenerated oil, in particular a thermally degenerated attached thereto by spraying or applying.

For the method of bringing the bactericide composition into contact with the hard surface with an oil spots or the like attached thereto, spraying or applying is preferred, and a method of spraying in the form of droplets or applying in the form of foam is preferred. Specifically, spraying means is used. That is, use of a bactericide article for a hard surface wherein the bactericide composition of the present invention is filled in a container having a sprayer is preferred. The present invention provides a bactericide article for a hard surface contained in a spray container wherein the bactericide composition of the present invention is filled in a container having a sprayer.

In the bactericide article for a hard surface contained in a spray container of the present invention, the container having a sprayer having the bactericide composition of the present invention filled therein includes a manual spray device such as a trigger-type spray container and a pump-type spray container, which does not use a propellant, or an aerosol using a propellant. The container having a sprayer is preferably a trigger-type spray that can spray or apply contents in the form of droplets or foam, more preferably a trigger-type spray having a mechanism for spraying contents in the form of droplets or a trigger-type spray having a mechanism (foam-forming mechanism) for forming foam.

For the bactericide article for a hard surface contained in a spray container of the present invention, when a trigger-type spray having a mechanism for spraying the bactericide composition of the present invention in the form of droplets is used, a spray nozzle of the spray container containing the composition has a diameter of spray hole in such a range of preferably 0.1 mm or more, more preferably 0.3 mm or more, and preferably 2 mm or less and more preferably 1 mm or less that spraying is easy, sprayed droplets are not rough and not sprayed linearly and an area produced by spraying is not extremely small.

When a trigger-type spray having a mechanism for spraying in the form of droplets is used, the bactericide article for a hard surface contained in a spray container of the present invention enables one operation thereof to spray the composition in an amount of preferably 0.1 mL or more, and more preferably 0.3 mL or more; and preferably 5 mL or less, and more preferably 2 mL or less.

When a trigger-type spray having a foam-forming mechanism is used, the bactericide article for a hard surface contained in a spray container of the present invention enables one operation thereof to spray the composition in an amount of preferably 0.5 mL or more, and more preferably 1 mL or more; and preferably 30 mL or less, more preferably 15 mL or less, and further preferably 5 mL or less.

The method for sterilizing a hard surface of the present invention is preferred as a method for sterilizing a hard surface of a hard article, further a hard surface of a food processing installation and/or a cooking installation. In addition, it is preferred as a sterilization method for washing by hand a hard surface of a hard article, further a sterilization method for washing by hand a hard surface of a food processing installation and/or a cooking installation.

The method for sterilizing a hard surface of the present invention regards, as an object to be sterilized, a hard surface of a hard article, preferably a hard surface of a food processing installation and/or a cooking installation.

Examples of the hard article, and the food processing installation and/or the cooking installation include those described above.

Examples of a material of a hard surface targeted by the method for sterilizing a hard surface of the present invention include plastics (including a silicone resin and the like), metal, earthenware, wood, and combinations thereof.

In the method for sterilizing a hard surface of the present invention, the bactericide composition of the present invention is brought into direct contact with a hard surface. Then, the hard surface may be allowed to stand in a state where the composition comes into contact, and therefore, during sterilization, it is not necessary to apply an external force such as washing by scrubbing with a flexible material such as a sponge.

The method for sterilizing a hard surface of the present invention may include a step of rinsing with water the hard surface, to which the bactericide composition has been brought into contact, preferably a step of rinsing with water after allowing the hard surface to stand in contact with the bactericide composition.

### EXAMPLES

Bactericide compositions shown in Tables 1 to 4 were prepared and used for the following evaluation. All the compositions had a pH at 20° C. of 7. Results are shown in Tables 1 to 4.

(1) 10 mL of LB medium was fed into a tissue culture flask, and bacteria (Staphylococcus aureus or colon bacilli) cultured in a petri dish were picked up with a platinum loop, and fed onto the LB medium.
(2) The flask was cultured at 200 rpm and 37° C. for 16 hours.
(3) The LB medium was moved to a 50 mL-centrifugation tube and centrifuged at 4000 rpm and 15° C. for 10 minutes.
(4) A supernatant of the LB medium was discarded, 10 mL of physiological saline solution was added, and centrifugation was carried out again under the same conditions as in (3).
(5) A supernatant of the physiological saline solution was discarded, physiological saline solution was added, so that an OD 1 bacterial suspension was produced.
(6) The solution produced in (5) was used to produce an OD 0.001 bacterial suspension with 0.5% BSA.
(7) 0.3 mL of the bacterial suspension produced in (6) was added, and uniformly applied and spread out onto a SUS304 plate with 8 cm×3 cm×0.1 cm.
(8) 0.3 mL of a bactericide composition was added, and uniformly applied and spread out onto the plate prepared in (7), and the plate was allowed to stand for 30 minutes.
(9) The solution remaining on the plate was poured into a swab having 1 mL of LP stop solution sealed therein, and a surface of the plate was swabbed and wiped with the swab.
(10) The solution was taken out from the swab, and serially diluted at intervals of 10 times.
(11) 0.1 mL of the serially-diluted solution was added to a standard agar medium, and applied and spread out with a bacteria spreader.
(12) The medium produced in (11) was cultured at 37° C. for 24 hours, and the colony count thereof was carried out to provide a viable cell count [Log (cfu/mL)].

TABLE 1

| | | | | Comparative Example | | | | | | | Example | |
|---|---|---|---|---|---|---|---|---|---|---|---|---|
| | | | | 1-1 | 1-2 | 1-3 | 1-4 | 1-5 | 1-6 | 1-7 | 1-1 | 1-2 |
| Bactericide | Formulation | (a) | a-1 | | 0.01 | | | | 0.02 | 0.01 | 0.01 | 0.01 |
| composition | (mass %) | (b) | b-1 | | | 0.01 | 0.01 | | | | 0.01 | 0.01 |
| | | | b-2 | | | | | | | | | |
| | | | b-3 | | | | | 0.01 | | | | |
| | | (b′) | b′-1 | | | | | | | 0.01 | | |

| | | | | | | | | | |
|---|---|---|---|---|---|---|---|---|---|
| Water | Balance | Balance | Balance | Balance | Balance | Balance | Balance | Balance | Balance |
| Total | 100 | 100 | 100 | 100 | 100 | 100 | 100 | 100 | 100 |

| | | | | | | | | | |
|---|---|---|---|---|---|---|---|---|---|
| (a)/(b)(mass ratio) | — | — | — | — | — | — | — | 1 | 1 |

| | | | | | | | | | |
|---|---|---|---|---|---|---|---|---|---|
| Viable cell count (Log(cfu/mL)) | 4.96 | 4.93 | 4.43 | 4.43 | 4.69 | 4.86 | 4.87 | <1 | 3.34 |
| (Staphylococcus aureus) | | | | | | | | | |

| | | | | Example | | | | | |
|---|---|---|---|---|---|---|---|---|---|
| | | | | 1-3 | 1-4 | 1-5 | 1-6 | 1-7 | 1-8 |
| Bactericide | Formulation | (a) | a-1 | 0.01 | 0.0075 | 0.00625 | 0.005 | 0.00375 | 0.0025 |
| composition | (mass %) | (b) | b-1 | | 0.0025 | 0.00375 | 0.005 | 0.00625 | 0.0075 |
| | | | b-2 | | | | | | |
| | | | b-3 | 0.01 | | | | | |
| | | (b′) | b′-1 | | | | | | |

| | | | | | | |
|---|---|---|---|---|---|---|
| Water | Balance | Balance | Balance | Balance | Balance | Balance |
| Total | 100 | 100 | 100 | 100 | 100 | 100 |

| | | | | | | |
|---|---|---|---|---|---|---|
| (a)/(b)(mass ratio) | 1 | 3 | 1.7 | 1 | 0.6 | 0.33 |

| | | | | | | |
|---|---|---|---|---|---|---|
| Viable cell count (Log(cfu/mL)) | 3.43 | 3.78 | 3.13 | 2.67 | 2.59 | 2.53 |
| (Staphylococcus aureus) | | | | | | |

TABLE 2

| Comparative Example | | | | Example | | |
|---|---|---|---|---|---|---|
| 2-1 | 2-2 | 2-3 | 2-4 | 2-1 | 2-2 | 2-3 |

| | | | | | | | | | |
|---|---|---|---|---|---|---|---|---|---|
| Bactericide | Formulation | (a) | a-2 | 0.01 | 0.02 | 0.01 | 0.01 | 0.01 | 0.01 |
| composition | (mass %) | (b) | b-1 | | | | 0.01 | | |
| | | | b-2 | | | | | 0.01 | |
| | | | b-3 | | | | | | 0.01 |
| | | (b′) | b′-1 | | | 0.01 | | | |

| | | | | | | | |
|---|---|---|---|---|---|---|---|
| Water | Balance | Balance | Balance | Balance | Balance | Balance | Balance |
| Total | 100 | 100 | 100 | 100 | 100 | 100 | 100 |

| | | | | | | | |
|---|---|---|---|---|---|---|---|
| (a)/(b) (mass ratio) | — | — | — | — | 1 | 1 | 1 |

| | | | | | | | |
|---|---|---|---|---|---|---|---|
| Viable cell count (Log(cfu/mL)) | 4.96 | 3.29 | 2.86 | 3.23 | 2.08 | 2.18 | 1.30 |
| (Staphylococcus aureus) | | | | | | | |

TABLE 3

| | | | | Comparative | | | | |
|---|---|---|---|---|---|---|---|---|
| | | | | Example | Example | | | |
| | | | | 3-1 | 3-1 | 3-2 | 3-3 | 3-4 |
| Bactericide | Formulation | (a) | a-2 | 0.06 | 0.03 | 0.0225 | 0.015 | 0.0066 |
| composition | (mass %) | (b) | b-3 | | 0.03 | 0.0375 | 0.045 | 0.0534 |

| | | | | | |
|---|---|---|---|---|---|
| Water | Balance | Balance | Balance | Balance | Balance |
| Total | 100 | 100 | 100 | 100 | 100 |

| | | | | | |
|---|---|---|---|---|---|
| (a)/(b) (mass ratio) | — | 1 | 0.6 | 0.33 | 0.12 |

| | | | | | |
|---|---|---|---|---|---|
| Viable cell count (Log(cfu/mL)) | 2.81 | 1.48 | <1 | 1.30 | 1.65 |
| (colon bacilli) | | | | | |

TABLE 4

| | | | | Comparative | |
|---|---|---|---|---|---|
| | | | | Example | Example |
| | | | | 4-1 | 4-1 |
| Bactericide | Formulation | (a) | a-1 | 0.06 | 0.0525 |
| composition | (mass %) | (b) | b-1 | | 0.0075 |

| | | |
|---|---|---|
| Water | Balance | Balance |
| Total | 100 | 100 |

| | | |
|---|---|---|
| (a)/(b) (mass ratio) | — | 7 |

| | | |
|---|---|---|
| Viable cell count(Log(cfu/mL)) (colon bacilli) | 2.37 | 1.23 |

In the tables, the expression “<1.0” indicates that the result was a detection limit or less.

In addition, the components in the tables are as follows.
a-1: lauryl dimethylamine oxide [a compound represented by the general formula (a1), wherein R^{1a} is a lauryl group, R^{2a} and R^{1a} are each a methyl group, and q and p are each 0]
a-2: myristyl dimethylamine oxide [a compound represented by the general formula (a1), wherein R^{1a} is a myristyl group, R^{2a} and R^{1a} are each a methyl group, and q and p are each 0]
b-1: polyoxyethylene hydrogenated tallow amine (average number of added moles: 2.1) [a compound represented by the general formula (b1), wherein R^{1b} is a hydrogenated tallow alkyl group and m+n is 2.1]
b-2: polyoxyethylene hydrogenated tallow amine (average number of added moles: 8) [a compound represented by the general formula (b1), wherein R^{1b} is a hydrogenated tallow alkyl group and m+n is 8]
b-3: polyoxyethylene cocoamine (average number of added moles: 2.1) [a compound represented by the general formula (b1), wherein R^{1b} is a coco alkyl group and m+n is 2.1]
b′-1: polyoxyethylene hydrogenated tallow amine (average number of added moles: 20) [a compound represented by the general formula (b1), wherein R^{1b} is a hydrogenated tallow alkyl group and m+n is 20]

1. A bactericide composition comprising:
(A) an alkyl or an alkenyl amine oxide having an alkyl group or an alkenyl group with 7 or more and 18 or less carbons (hereinafter referred to as a component (a)); and
(B) a mono- or polyoxyalkylene alkyl or alkenyl amine having an alkyl group or an alkenyl group with 8 or more and 24 or less carbons and including oxyethylene group as an oxyalkylene group, wherein an average number of added moles of the oxyethylene group is one or more and 8 or less (hereinafter referred to as a component (b)).

2. The bactericide composition according to claim 1, wherein the component (a) is contained in an amount of 0.00005% by mass or more and 5% by mass or less.

3. The bactericide composition according to claim 1, wherein a mass ratio (a)/(b) of the content of the component (a) and the content of the component (b) is 0.001 or more and 10 or less.

4. The bactericide composition according to claim 1, wherein a ratio of the total of the content of the component (a) and the content of the component (b) is 70% by mass or more relative to the total content of surfactants.

5. A method of enhancing a bactericidal effect of a bactericide composition, wherein the bactericidal effect of the bactericide composition is enhanced by adding, to (a) an alkyl or an alkenyl amine oxide having an alkyl group or an alkenyl group with 7 or more and 18 or less carbons, (b) a mono- or polyoxyalkylene alkyl or alkenyl amine having an alkyl group or an alkenyl group with 8 or more and 24 or less carbons and including oxyethylene group as an oxyalkylene group, wherein an average number of added moles of the oxyethylene group is one or more and 8 or less.

6. A sterilization method wherein the bactericide composition according to claim 1 is brought into contact with a hard surface.

7. The sterilization method according to claim 6, wherein the bactericide composition contains the component (a) in an amount of 0.00005% by mass or more and 0.5% by mass or less.
